(19) United States
(12) Patent Application Publication
(10) Pub. No.: US 2024/0185567 A1
(43) Pub. Date: Jun. 6, 2024

(54) SYSTEM AND METHOD FOR OUTCOME EVALUATIONS ON HUMAN IVF-DERIVED EMBRYOS

(71) Applicant: Kang ZHANG, Del Mar, CA (US)

(72) Inventor: Kang ZHANG, Del Mar, CA (US)

(21) Appl. No.: 18/388,515

(22) Filed: Nov. 9, 2023

Related U.S. Application Data
(63) Continuation of application No. PCT/US22/28553, filed on May 10, 2022.
(60) Provisional application No. 63/186,179, filed on May 10, 2021.

(51) Int. Cl. G06V 10/764; G06T 7/00; G06T 7/68; G16H 50/20

(52) U.S. Cl. CPC G06V 10/764; G06T 7/0016; G06T 7/68; G16H 50/20; G06T 2207/10016; G06T 2207/20081; G06T 2207/20084; G06T 2207/30024; G06V 2201/03

(57) ABSTRACT

AI-based method and system are provided for embryo morphological grading, blastocyst embryo selection, aneuploidy prediction, and final live birth outcome prediction in In vitro fertilization (IVF). The method and system can employ deep learning models based on image data of one or more human embryos, where the image data include a plurality of images of the one or more human embryo at different time points within the first few days after the formation of the one or more embryos.

### CROSS REFERENCE TO RELATED APPLICATION

This application is a continuation of International Application No. PCT/US22/28553 filed May 10, 2022, which claims the benefit of U.S. Provisional Application No. 63186179, filed May 10, 2021, the disclosure of each of which is incorporated herein by reference in its entirety.

### BACKGROUND

More than 80 million couples suffer from infertility. In vitro fertilization (IVF) has revolutionized treatment for infertility in which more than 5 million babies have been born from IVF. However, to achieve a favorable live birth outcome is still challenging. Traditional methods of embryo selection depend on visual inspection of embryo morphology and are experience-dependent and highly variable¹⁻³. An automated system that performs a complex task of a skilled embryologist and incorporates assessments such as zona pellucida thickness variation, number of blastomeres, degree of cell symmetry and cytoplasmic fragmentation, aneuploidy status, and maternal conditions to predict the final outcome of a live birth is highly desirable^{4,5}.

Artificial intelligence has the potential to revolutionize healthcare and improve outcomes⁶⁻⁹ in all areas, such as image-based diagnosis¹⁰, voice recognition, and natural language processing¹¹. In particular, the use of convolutional neural networks with transfer learning has facilitated efficient and accurate image diagnosis^{10,12}.

Application of deep learning in IVF has been explored in classifying embryos based on morphological quality or and transfer outcomes, although their accuracies and general applicability remain to be a major challenge^{4,5,13-16}. Furthermore, sub-optional outcome predictions based on traditional human performance severely limits the impact of the IVF technology, particularly in resource and access poor areas^{17,18} An Ai algorithm capable of assessing and ranking embryos for implantation, and combining maternal metrics to predict live birth outcomes have great utility.

Preimplantation genetic testing (PGT) for the detection of aneuploidy has improved the success rate of embryo transfer and pregnancy outcomes. However, it has several limitations including invasiveness, the cost of sequencing, mosaicism, experience in trophectoderm biopsy.

### SUMMARY OF THE INVENTION

In one aspect, the present disclosure provides a computer-implemented method comprising the steps of: receiving image data of one or more human embryos, the image data including a plurality of images of the one or more human embryo at different time points within the first 6 days of the formation of the one or more embryos: determining a viability indicator for the one or more human embryos, wherein the viability indicator represents a likelihood that selection for implantation of the one or more embryos will result in a viable embryo, based on one or more the following: by using at least one computer processor, determining embryo morphological grading of the one or more embryos using a first neural network based on the image data: by using at least one computer processor, determining aneuploidy of the one or more embryos using a second deep learning model at least partly based on the image data: by using at least one computer processor, predicting live-birth occurrence of a transfer of the one or more embryos for implantation using a third deep learning model at least partly based on the image data: and outputting the viability indicator.

In some embodiments, determining the embryo morphological grading comprises using a multitask machine learning model based on the following three tasks: (1) a regression task for the cytoplasmic fragmentation rate of the embryo, (2) a binary classification task for the number of cells of the embryo, and (3) a binary classification task for the blastomere asymmetry of the embryo determined, based on the image data. In some embodiments, the multitask machine learning model was trained jointly through combining the loss functions of the three tasks by using a homoscedastic uncertainty approach in minimizing the joint loss. In some embodiments, output parameters for the embryo morphological grading comprise pronucleus type on Day 1, the number of blastomeres, asymmetry, and fragmentation of blastomeres on Day 3.

In some embodiments, determining the viability indicator comprises determining aneuploidy of the one or more embryos using the second deep learning model at least partly based on the image data. In some embodiments, determining the viability indicator comprises predicting live-birth occurrence of a transfer of the one or more embryos for implantation using the third deep learning model at least partly based on the image data.

In some embodiments, determining a viability indicator for the human embryo further comprises using clinical metadata of the donor of the egg from the embryo is developed, the metadata includes at least one of maternal age, menstrual status, uterine status, and cervical status, previous pregnancy, and fertility history.

In some embodiments, the second deep learning model in the aneuploidy determination comprises a 3D CNN model trained by time-lapse image videos and PGT-A based ploidy outcomes assessed by biopsy.

In some embodiments, the method further comprises: determining blastocyst formation based on the embryo image data based on Day 1 and Day 3.

In some embodiments, the third deep learning model comprises a CNN model. In some embodiments, the third deep learning model can further comprise an RNN model, and a two-layer perceptron classifier.

In some embodiments, the method further includes: determining a ranking of a plurality of human embryos based on their viability indicators.

In some embodiments, the method further includes: selecting, based on the ranking, one of the plurality of human embryos for a single embryo transfer or the order in which multiple embryos should be transferred.

In some embodiments, the method further comprises selecting the embryo for transfer and implantation based on the determined viability indicator. The selection for transfer and implantation can be on Day 3, Day 5/6.

In another aspect, the present disclosure provides a method of selecting a human embryo in an IVF/ICSI cycle, which includes determining a viability indicator using a computer-implemented prediction method described herein, and based on the predicted viability indicator, selecting the human embryo for transfer and implantation.

In another aspect, the present disclosure provides a system, including at least one processor configured to: receive image data of one or more human embryos, the image data including a plurality of images of the one or more human embryos with at different time points within the first 6 days after the formation of the one or more embryos: apply at least one three-dimensional (3D) artificial neural network to the image data to determine a viability indicator for the one or more human embryos: and output the viability score.

### BRIEF DESCRIPTION OF THE DRAWINGS

FIG. 1 is a schematic illustration of an embodiment of the disclosed AI platform for embryo assessment and live-birth occurrence prediction during the whole IVF circle.

FIG. 2 shows performance in the evaluation of embryos' morphokinetic features according to embodiments of the disclosed subject matter.

FIG. 3 shows performance in predicting the development to the blastocyst stage according to embodiments of the disclosed subject matter.

FIG. 4 shows performance of certain embodiments of the disclosed subject matter in identifying blastocyst ploidy (euploid/aneuploid).

FIG. 5 shows performance of certain embodiments of the disclosed subject matter in predicting live-birth occurrence of disclosed AI models.

FIG. 6 shows visualization of evidence for embryo morphological assessment according to embodiments of the disclosed subject matter.

FIG. 7 is a flowchart of an embodiment of the disclosed AI platform with an ensemble of model instances.

FIG. 8 is a flow diagram describing the datasets of embodiments of the disclosed subject matter.

FIG. 9 shows performance in the measurement of embryos' morphokinetic features according to embodiments of the disclosed subject matter.

FIG. 10 shows performance in predicting the development to the blastocyst stage according to embodiments of the disclosed subject matter.

FIG. 11 shows performance study of the live-birth occurrence of certain embodiments of the disclosed subject matter.

FIG. 12 schematically illustrates a computer control system or platform that is programmed or otherwise configured to implement methods provided herein.

### DESCRIPTION OF CERTAIN EMBODIMENTS OF THE INVENTION

According to some aspects, disclosed herein are diagnostic systems, computing devices, and computer-implemented methods to evaluate embryos generated by IVF procedures, such as embryo ploidy and live birth occurrence probability, by using a machine learning framework and without using biopsy. In some embodiments, the machine learning framework utilizes deep learning models such as neural networks.

In one aspect, the present disclosure provides a method of selecting euploidy embryos based on a deep learning method using spatial and temporal information stored in time-lapse images. These images with corresponding parameters may store information corresponding genetic information underlying proper embryo development, therefore amendable to an AI based prediction on embryo ploidy (euploid vs. aneuploid) without a biopsy.

Embodiments of the present invention provide a method for estimating embryo viability. The viability indicator is or can include a probability, providing a prediction of the likelihood of an embryo leading to a successful pregnancy after implantation in the uterus. The embryo with a higher value of viability indicator has a higher probability of pregnancy and live-birth. If multiple embryos are to be transferred, the viability score may be used to decide the order in which embryos will be transferred into the uterus.

In one aspect, the present disclosure provides a computer-implemented method comprising the steps of: receiving image data of one or more human embryos, the image data including a plurality of images of the one or more human embryo at different time points within the first 6 days of the formation of the one or more embryos: determining a viability indicator for the one or more human embryos, wherein the viability indicator represents a likelihood that selection for implantation of the one or more embryos will result in a viable embryo, based on one or more the following: determining embryo morphological grading of the one or more embryos using a first neural network based on the image data: determining aneuploidy of the one or more embryos using a second deep learning model at least partly based on the image data: predicting live-birth occurrence of a transfer of the one or more embryos for implantation using a third deep learning model at least partly based on the image data: and outputting the viability indicator.

In some embodiments, determining the embryo morphological grading comprises using a multitask machine learning model based on the following three tasks: (1) a regression task for the cytoplasmic fragmentation rate of the embryo, (2) a binary classification task for the number of cells of the embryo, and (3) a binary classification task for the blastomere asymmetry of the embryo determined, based on the image data.

In some embodiments, the multitask machine learning model was trained jointly through combining the loss functions of the three tasks by using a homoscedastic uncertainty approach in minimizing the joint loss.

In some embodiments, output parameters for the embryo morphological grading comprise pronucleus type on Day 1, the number of blastomeres, asymmetry, and fragmentation of blastomeres on Day 3.

In some embodiments, determining a viability indicator for the human embryo further comprises using clinical metadata of the donor of the egg from the embryo is developed, the metadata includes at least one of maternal age, menstrual status, uterine status, and cervical status, previous pregnancy, and fertility history.

In some embodiments, the second deep learning model in the aneuploidy determination comprises a 3D CNN model trained by time-lapse image videos and PGT-A based ploidy outcomes assessed by biopsy.

In some embodiments, the method further comprises: determining blastocyst formation based on the embryo image data based on Day 1 and Day 3.

In some embodiments, the third deep learning model comprises a CNN model. In some embodiments, the third deep learning model further comprises an RNN model and a two-layer perceptron classifier.

In some embodiments, the method further includes: determining a ranking of a plurality of human embryos based on their viability indicators.

In some embodiments, the method further includes: selecting, based on the ranking, one of the plurality of human embryos for a single embryo transfer or the order in which multiple embryos should be transferred.

In some embodiments, the method further comprises selecting the embryo for transfer and implantation based on the determined viability indicator. The selection for transfer and implantation can be on Day 3, Day 5/6.

In another aspect, the present disclosure provides a method of selecting a human embryo in an IVF/ICSI cycle, which includes determining a viability indicator of one or more IVF-derived embryos using a computer-implemented prediction method described herein, and based on the predicted viability indicator, selecting a human embryo for transfer and implantation.

In another aspect, the present disclosure provides a system or device including at least one processor, a memory, and non-transitory computer readable storage media encoded with a program including instructions executable by the at least one processor and cause the at least one processor to: receive image data of one or more human embryos, the image data including a plurality of images of the one or more human embryos with at different time points within the first 6 days after the formation of the one or more embryos: apply at least one three-dimensional (3D) artificial neural network to the image data to determine a viability indicator for the one or more human embryos: and output the viability score.

In some embodiments, the systems, devices. media, methods and applications described herein include a digital processing device. For example, in some embodiments, the digital processing device is part of a point-of-care device integrating the diagnostic software described herein. In some embodiments, the medical diagnostic device comprises imaging equipment such as imaging hardware (e.g. a camera) for capturing medical data (e.g. medical images). The equipment may include optic lens and/or sensors to acquire images at hundreds or thousands of magnification. In some embodiments, the medical imaging device comprises a digital processing device configured to perform the methods described herein. In further embodiments, the digital processing device includes one or more processors or hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device. In accordance with the description herein, suitable digital processing devices include, by way of non-limiting examples, server computers, desktop computers, laptop computers, notebook computers, sub-notebook computers, netbook computers, set-top computers, handheld computers, Internet appliances, mobile smartphones, tablet computers, personal digital assistants, video game consoles, and vehicles. Those of skill in the art will recognize that many smartphones are suitable for use in the system described herein.

In some embodiments, the system, media. methods and applications described herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory. magnetic disk drives, magnetic tape drives, optical disk drives, cloud computing systems and services, and the like. In some cases, the program and instructions are permanently, substantially permanently, semi-permanently, or non-transitorily encoded on the media.

In some embodiments, the system, media. methods and applications described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein. those of skill in the art will recognize that a computer program may be written in various versions of various languages.

The functionality of the computer readable instructions may be combined or distributed as desired in various environments. In some embodiments, a computer program comprises one sequence of instructions. In some embodiments. a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof. In some embodiments, a computer program includes a web application. In light of the disclosure provided herein, those of skill in the art will recognize that a web application, in various embodiments, utilizes one or more software frameworks and one or more database systems.

In some embodiments, the systems, devices, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments. software modules are in one computer program or application. In other embodiments, software modules are in more than one computer program or application. In some embodiments, software modules are hosted on one machine. In other embodiments, software modules are hosted on more than one machine. In further embodiments. software modules are hosted on cloud computing platforms. In some embodiments, software modules are hosted on one or more machines in one location. In other embodiments, software modules are hosted on one or more machines in more than one location

### DETAILED FIGURE DESCRIPTIONS

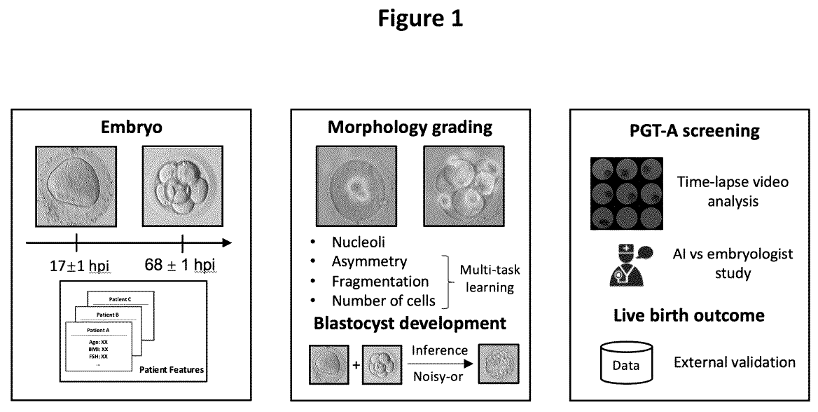

FIG. 1. Schematic illustration of disclosed AI platform for embryo assessment and live-birth occurrence prediction during the whole IVF circle.

The left panel: The AI models utilized images of human embryos captured at 17±1 hours post-insemination (the Day 1) or 68±1 hours post-insemination (the Day 3). Clinical metadata (e.g., maternal age, BMI) are also included.

The middle and right panel: An illustration of the explainable deep-learning system for embryo assessment during the whole IVF circle. The system consisted of four modules. The middle panel: a module for grading embryo morphological features using multitask learning: a module for blastocyst formation prediction using Day 1/Day 3 images with noisy-or inference.

The right panel: a module for predicting embryo ploidy (euploid vs. aneuploid) using embryo images or time-lapse videos: a final module for the live-birth occurrence prediction using images and clinical metadata. The models were tested on independent cohorts to ensure the generalizability. We also studied the AI versus embryologist comparison performance.

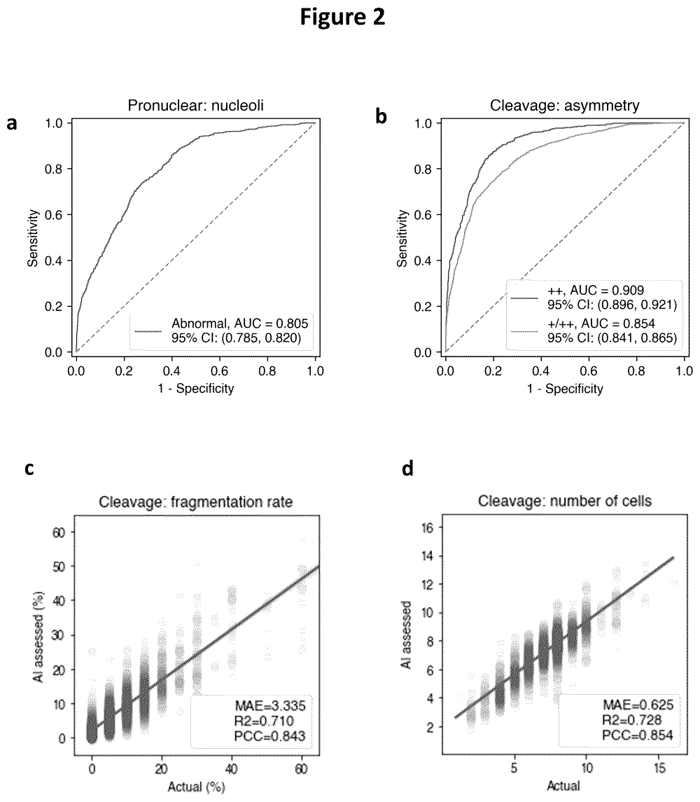

FIG. 2. Performance in the evaluation of embryos' morphokinetic features using disclosed AI system.

a, ROC curve showing performance of detecting abnormal pronucleus type of the Day 1 embryo. b-d, Morphological assessment of the D3 embryos. b, ROC curves showing performance of detecting blastomere asymmetry. The orange line represents detecting asymmetry (++or +) from normal (−). The blue line represents detecting severe asymmetry (++) from good one (−). c, Correlation analysis of the predicted embryo fragmentation rate versus the actual embryo fragmentation rate. d, Correlation analysis of the predicted blastomere cell number versus the actual blastomere cell number. MAE, mean absolute error: R2, coefficient of determination: PCC, Pearson's correlation coefficient.

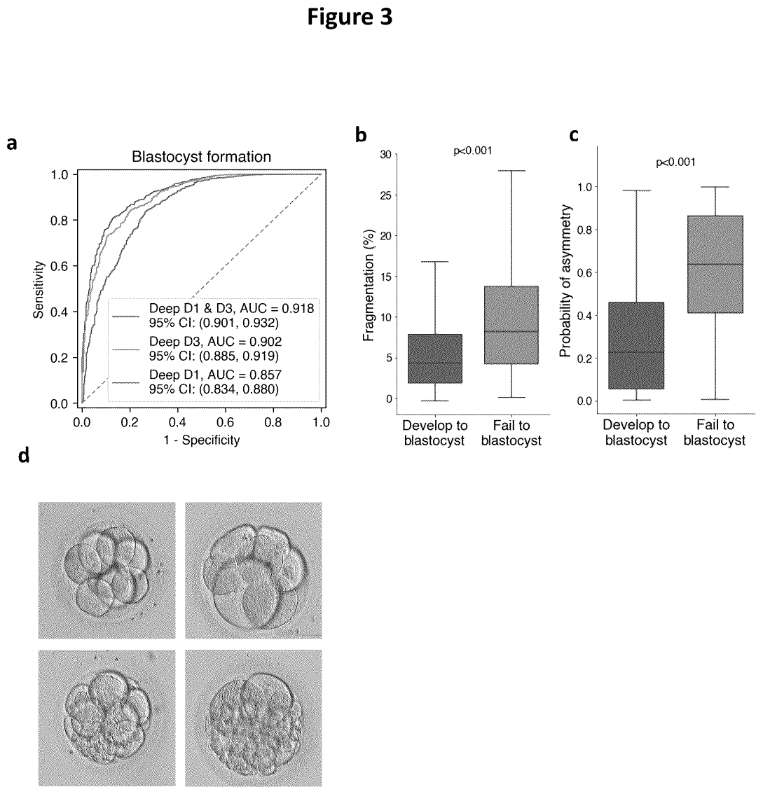

FIG. 3. Performance in predicting the development to the blastocyst stage using disclosed AI system.

a, ROC curves showing performance of selecting embryos that developed to the blastocyst stage. The blue, orange, and green lines represent using images from Day 1, Day 3 and combined Day 1 & Day3, respectively.

b-d, The morphology of embryos is positively related to blastocyst development including, b, embryo fragmentation rate, and c, blastomere asymmetry. Box plots showed median, upper quartile and lower quartile (by the box) and the upper adjacent and lower adjacent values (by the whiskers). d, Visualization for embryos' morphokinetic characteristics that developed to the blastocyst stage or not.

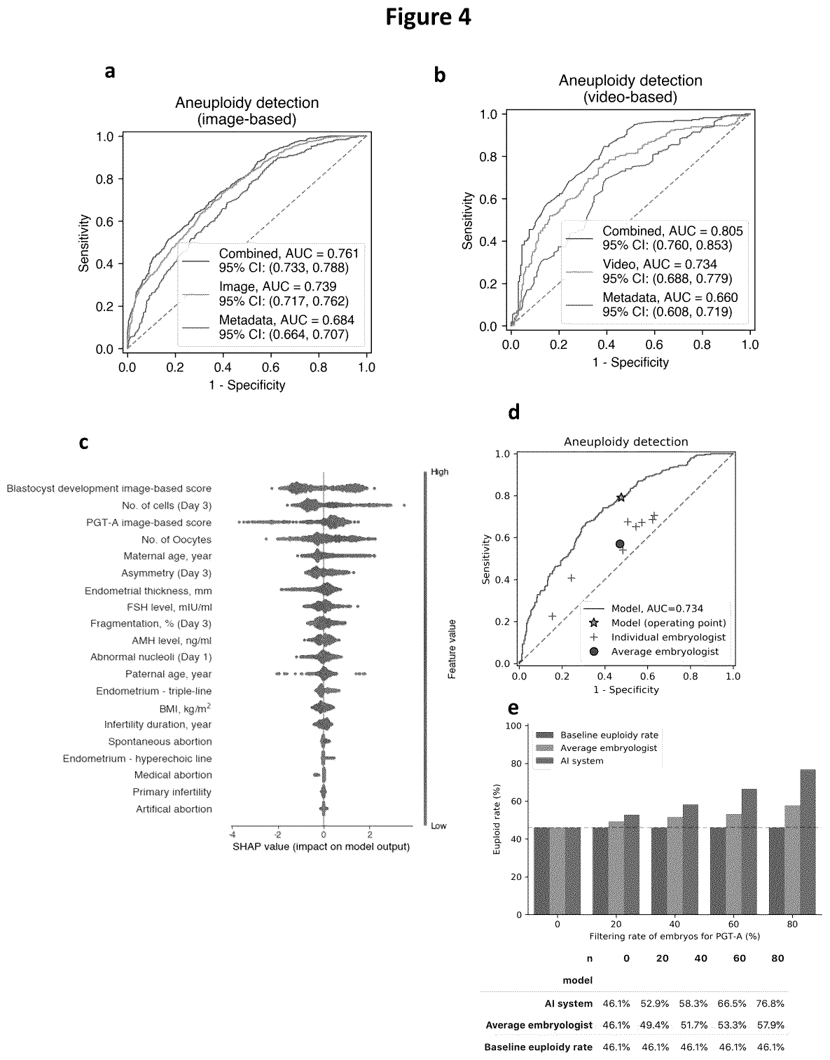

FIG. 4. Performance of disclosed AI system in identifying blastocyst ploidy (euploid/aneuploid)

a, The ROC curves for a binary classification using the clinical metadata-only model, the embryo image-only model and the combined model. PGT-A test results are available.

b, The ROC curves for a binary classification using the clinical metadata-only model, the embryo video-only model and the combined model. The videos of embryo development is captured using time-lapse.

c, Illustration of features contributing to progression to euploid blastocysts by SHAP values. Features on the right of the risk explanation bar pushed the risk higher and features on the left pushed the risk lower.

d and e, Performance comparison between our AI model and eight practicing embryologists in embryos' euploid ranking. d, ROC curves for detecting aneuploidy. Individual embryologist performance is indicated by the red crosses and averaged embryologist performance is indicated by the green dot. e, The euploid rate of blastocysts selected for PGT-A test by AI versus average embryologists on different filtering rate senerios. The baseline euploid rate is 46.1%

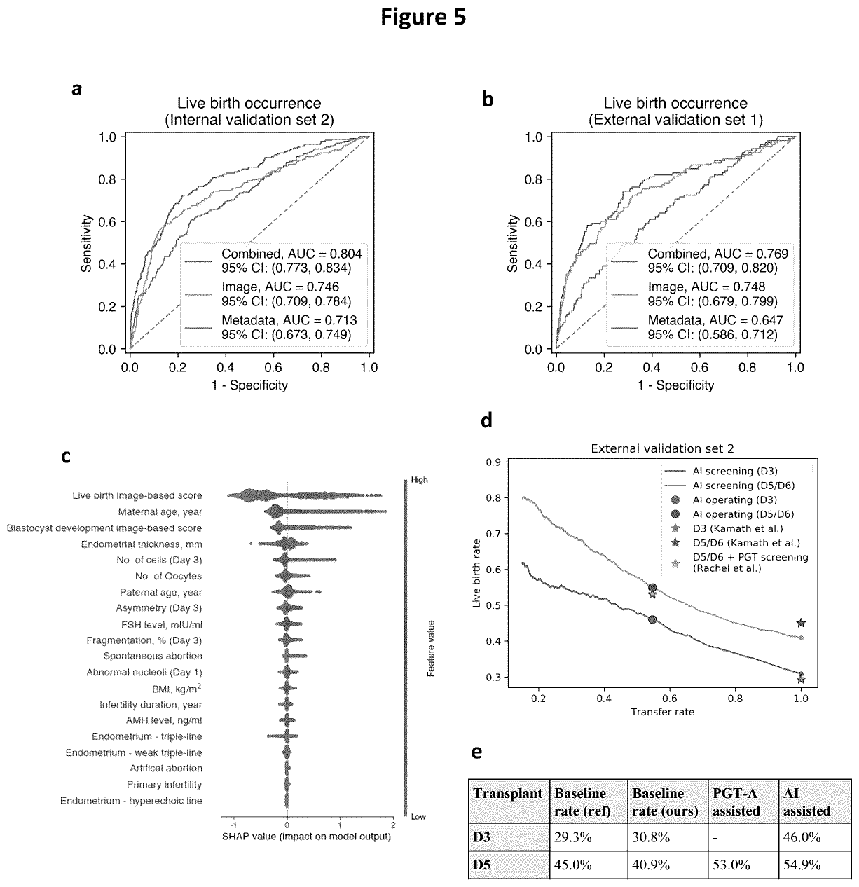

FIG. 5. Performance in predicting live-birth occurrence of disclosed AI models.

a and b, ROC curves showing performance of on live-birth occurrence prediction on, a, internal test set: b, external validation cohort. The orange, green and blue ROC curves represent using the metadata-only model, the embryo image-only model and the combined model.

c, Illustration of features contributing to progression to live-birth occurrence by SHAP values.

d and e, Comparison of our AI system with the PGT-A assisted approach for live-birth occurrence. d, The live birth rate by the AI system is associated with the proportion of embryos be selected for transfer. The orange line represents transplant on Day 3. The blue line represents transplant on Day 5/6. e, Illustration of the baseline rate by Kamath et.al., baseline rate on our external validation set 2, the PGT-A assisted live-birth rate and the AI-assisted live-birth rate. PGT-A is only performed for Day 5/6 transplant.

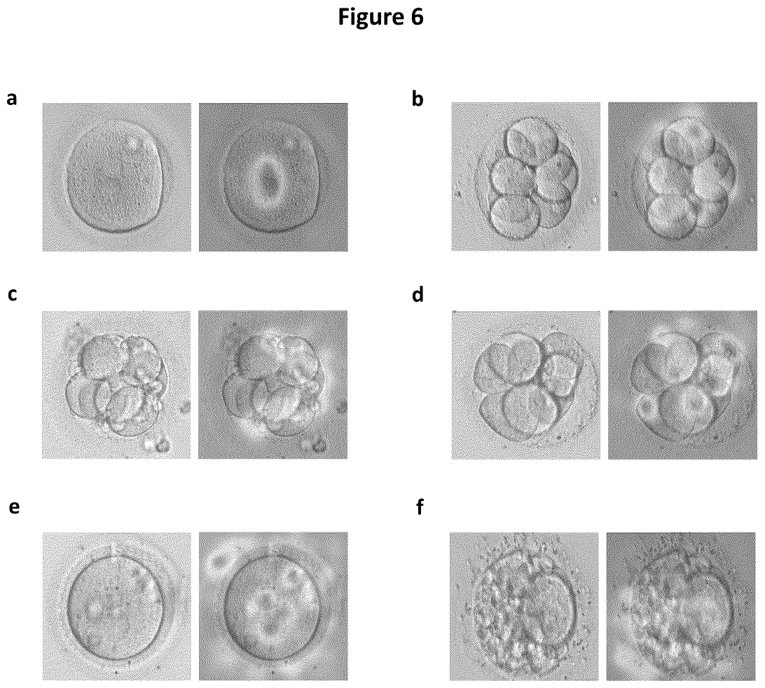

FIG. 6. Visualization of evidence for embryo morphological assessment using integrated gradients method.

Left: the original embryo images: Right: Explanation method generated saliency heatmaps. a, normal pronuclear type of Day I (good one): b, blastomere symmetry of Day3 (good one): c, fragmentation rate of Day3 embryo (normal): d, Day3 blastomere cell number (normal): e, Day 1 embryo failed to develop to the blastocyst stage: f, Day 3 embryo failed to develop to the blastocyst stage.

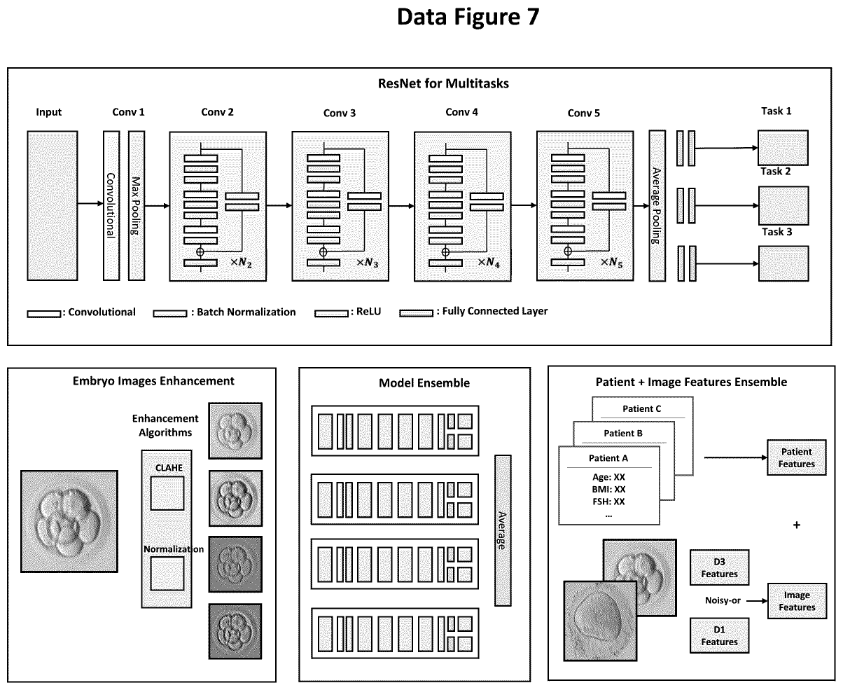

FIG. 7. The flowchart of the AI platform with an ensemble of model instances.

We first developed image enhancement models using color normalization and contrast-limited adaptive histogram equalization (CLAHE) techniques. Four types of embryo images after the application of color normalization and CLAHE image enhancements: original image, image after applying the CLAHE transformation only, image after applying the color normalization transformation only, and image after applying both the CLAHE and color normalization transformations. Each image instance separately makes a prediction, and these are combined by averaging the results for producing a robust AI model.

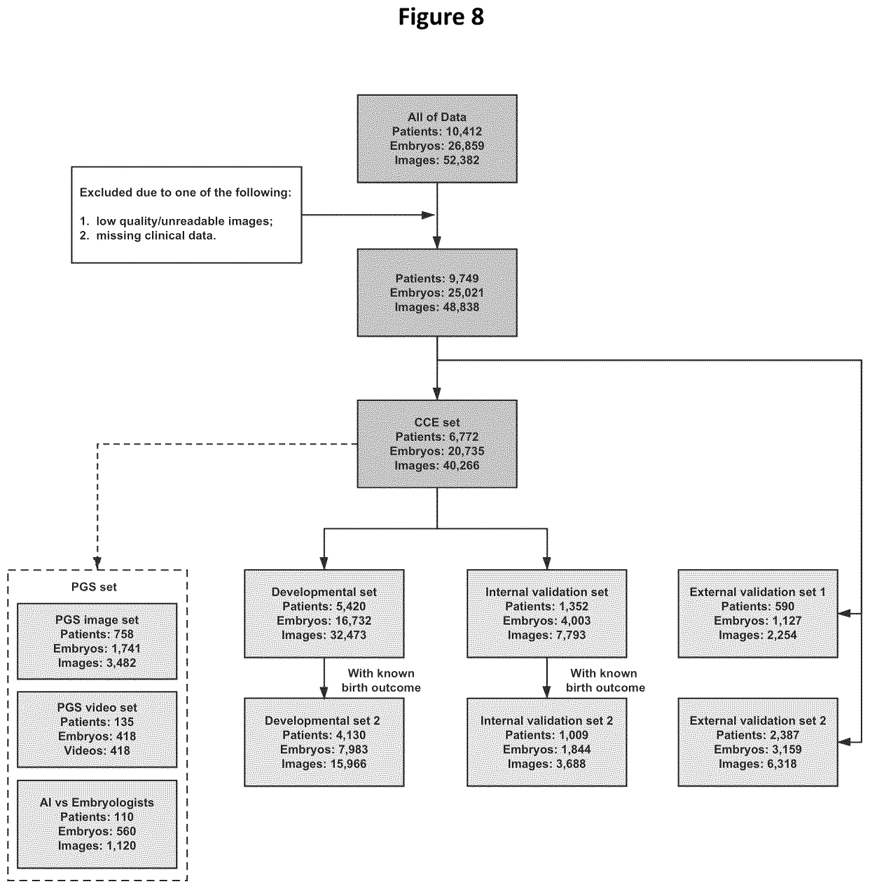

FIG. 8. Flow diagram describing the datasets used for disclosed AI system, including 4 principal modules: morphology grading, blastocysts prediction, PGT-A ranking, and live-birth occurrence prediction. Patient inclusion and exclusion criteria were also considered.

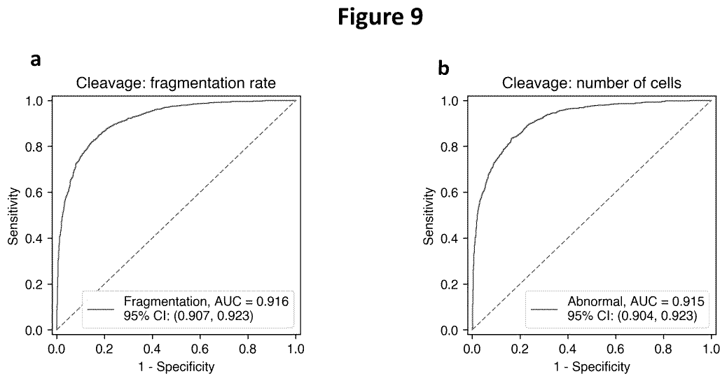

FIG. 9. Performance in the measurement of embryos' morphokinetic features using disclosed AI system. Relating to FIG. 2.

a and b, ROC curves showing performance of detecting abnormal morphology of the Day3 embryo. a, ROC curves showing performance of detecting fragmentation. b, ROC curve showing performance of identification of abnormal cell number (we defined the numbers 7-9 as normal, otherwise are abnormal)

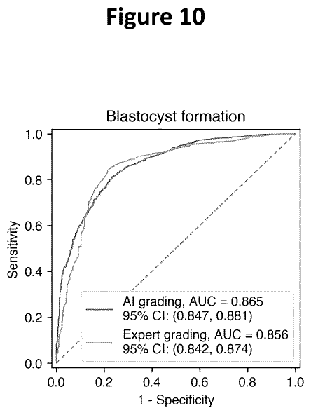

FIG. 10. Performance in predicting the development to the blastocyst stage using the AI system.

ROC curves showing performance of selecting embryos that developed to the blastocyst stage. The blue line represents using the morphological scores given by physicians: the orange line represents using the morphological scores given by our AI system.

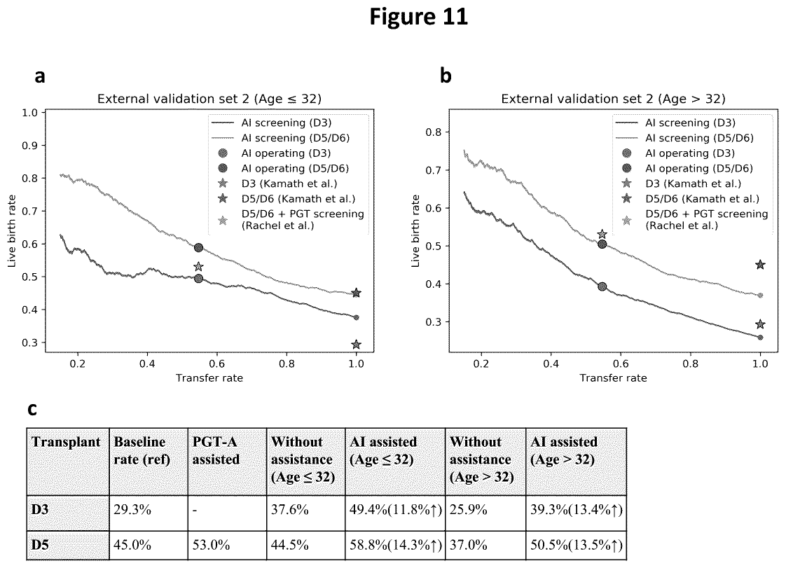

FIG. 11. Performance study of the live-birth occurrence of the AI models.

Comparison of our AI system with the PGT-A assisted approach for live-birth occurrence. a and b, The live birth rate by the AI system is associated with the proportion of embryos be selected for transfer. The orange line represents transplant on Day 3. The blue line represents transplant on Day 5/6. a, maternal age (≤32, median age): b, maternal age (>32, median age): c, Illustration of the baseline rate by Kamath et.al., baseline rate on our external validation set 2, the PGT-A assisted live-birth rate and the AI-assisted live-birth rate. PGT-A is only performed for Day 5/6 transplant.

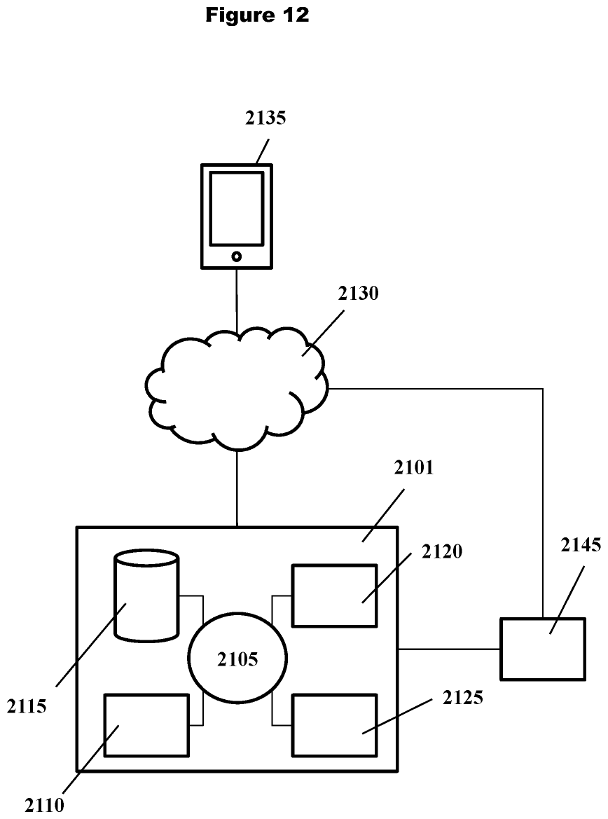

FIG. 12 schematically illustrates a computer control system or platform that is programmed or otherwise configured to implement methods provided herein. In some embodiments, the system comprises a computer system 2101 that is programmed or otherwise configured to carry out executable instructions such as for carrying out image analysis. The computer system includes at least one CPU or processor 2105. The computer system includes at least one memory or memory location 2110 and/or at least one electronic storage unit 2115. In some embodiments, the computer system comprises a communication interface 2120 (e.g. network adaptor). In some embodiments, the computer system 2101 can be operatively coupled to a computer network (“network”) 2130 with the aid of the communication interface 2120. In some embodiments, an end user device 2135 is used for uploading image data such as embryo images, general browsing of the database 2145, or performance of other tasks. In some embodiments, the database 2145 is one or more databases separate from the computer system 2101.

### EXAMPLE

An AI-based system was developed to cover the entire IVF/ICSI cycle, which consisted of four main components: an embryo morphological grading module, a blastocyst formation assessment module, an aneuploid detection module, and a final live-birth occurrence prediction module. Based on multitask learning, AI models were provided for embryo morphological assessment, including pronucleus type on day 1, and number of blastomeres, asymmetry and fragmentation of blastomeres on day 3. Several key issues in IVF were addressed, including embryo morphological grading, blastocyst embryo selection, aneuploidy prediction, and final live birth outcome prediction. Transfer learning were used to pre-train a CNN with 10 million ImageNet images and applied this model to D1/D3 human embryo images for further AI system development covering the whole IVF/ICSI cycle. The above two approaches enable us to assess implantation potential. Prediction on a live-birth outcome also depend on many factors including maternal age, factors involving in menstrual, uterine, and cervical status, previous pregnancy and fertility histories, which factors are also incorporated in the AI models herein. By combining with embryo and maternal metrics in an ensemble AI model, we evaluated live-birth outcomes in a prospective trial (See FIG. 1).

### Methods

### Dataset Characteristics

Data (embryo Images and medical records) were collected at Guangzhou Women and Children's Hospital and Jiangmen central hospital between 2010 and 2019.

This study was approved by the Reproductive Medical Ethics Committee of Guangzhou Women and Children's Hospital.

All procedures were performed as a part of a patients' standard care. Institutional Review Board (IRB)/Ethics Committee approvals were obtained in all locations and all participating subjects signed a consent form.

### Overview of IVF-ET Cycles

The oocytes were inseminated by conventional IVF or ICSI according to sperm parameter after retrieved. Then, all the two-pronuclei embryos were cultured individually after fertilization check, and they turned into cleavage stage embryo after cell division. The embryos were observed daily up to day-5/6 with each embryo has at least two photographs: at fertilization check (16-18 h after insemination) and Day-3 embryo assessment (66 h after insemination) (Extended Data Table 1 and 2).

### Extended Data Table 1. Observation of Fertilized Oocytes, Embryos, and Expected Stage of Development at Each Time Point Based on Istanbul Consensus

| | Timing | |
|---|---|---|
| | (hours post- | Expected stage |
| Type of observation | insemination) | of development |
| Fertilization check | 17 + 1 | Pronuclear stage |
| Day-2 embryo assessment | 44 + 1 | 4-cell stage |
| Day-3 embryo assessment | 68 + 1 | 8-cell stage |
| Day-4 embryo assessment | 92 + 2 | Morula |
| Day-5 embryo assessment | 116 + 2 | Blastocyst |
| Day-6 embryo assessment | 140 + 2 | |

### Extended Data Table 2. Morphology Assessment of Embryos

| | | Grade | Rating | Description |
|---|---|---|---|---|
| scoring system | | 1 | Symmetrical | Equivalent to Z1 and Z2 |
| for pronuclei | | 2 | Non- | Other arrangements, |
| | | | symmetrical | including peripherally |
| | | | | sited pronuclei |
| scoring system for | | 1 | Cells number | 4\5\6\7\8\9\10\compact |
| cleavage-stage embryos | | 2 | Symmetry of | −/+/++ |
| (D3 embryo) | | | cell size | |
| | | 3 | fragmentation | 0%~80% |
| | | 4 | multinucleation | Yes or no |
| scoring system | Stage of | 1 | Early blastocyst with a blastocoel that is less | |
| for blastocysts | development | | than half of the volume of the embryo | |
| | | 2 | a blastocyst with a blastocoel that is half of or | |
| | | | greater than half of the volume of the embryo | |
| | | 3 | a full blastocyst with a blastocoel completely | |
| | | | filling the embryo | |
| | | 4 | an expanded blastocyst with a blastocoel | |
| | | | volume larger than that of the early embryo, | |
| | | | with a thinning zona | |
| | | 5 | a hatching blastocyst with the trophectoderm | |
| | | | starting to herniate though the zona | |
| | | 6 | a hatched blastocyst, in which the blastocyst | |
| | | | has completely escaped from the zona | |
| | ICM | 1 | Good, A | Good Prominent, easily |
| | | | | discernible, with many |
| | | | | cells that are compacted |
| | | | | and tightly adhered |
| | | 2 | Fair, B | Easily discernible, with |
| | | | | many cells that are loosely |
| | | | | grouped together |
| | | 3 | Poor, C | Difficult to discern, with |
| | | | | few cells |
| | TE | 1 | Good, A | Good Many cells forming a |
| | | | | cohesive epithelium |
| | | 2 | Fair, B | Few cells forming a loose |
| | | | | epithelium |
| | | 3 | Poor, C | Very few cells |

For Day 1 (16-18 h later) embryo morphological evaluation, embryologist scored the zygote according to the number, size and location of the pronucleus and pronuclei. Scott et al.²⁸ classified zygotes into four groups Z1-Z4 according to pronuclear morphology labeled with grades corresponding to their quality, including nuclear size, nuclear alignment, nucleoli alignment and distribution and the position of the nuclei within the zygote.

Cleavage-stage embryos were evaluated by cell number, relative degree of fragmentation, and blastomere asymmetry, according to the Istanbul consensus (consensus 2011)²⁹.

If the embryo was cultured to blastocyst, Day-5 or Day-6 photograph were stored to analysis as well. Only available blastocysts (defined as stage≤3, and at least one score of inner cell mass or trophectoderm is ≥B) were selected for transferred or frozen for future use.

If the embryo was scheduled to PGT, biopsy was performed on day 5 or day 6 according to the blastocyst grade, and NGS was enrolled for euploidy assessment. In PGT cycles, all the embryos went on blastocyst culture, available blastocysts were biopsied and NGS was carried out for euploidy assessment.

Most of the embryos were transferred according to morphological scores on day 3 or blastocyst stage, while in PGT cycles embryos were selected according to PGT diagnosis reports.

All the patients were strictly followed up, and live birth was defined as the birth of a live infant at ≥28 weeks of gestation.

Time-lapse videos were also carried out in parts of the patients and were also used for analysis. We used images from the Primo Vision time lapse system, which takes an image for the embryoes every 10 minutes at 9 focal planes, at 10 μm increments.

### Embryo Scoring

Nine senior embryologists from the two centers scored embryos according to the internal scoring rules.

For embryos which have definitely results were included this study. Euploid embryos in retrospective study were included single embryo transfer results one live birth, or two embryos transfer results in twin babies.

Viability blastocyst is defined as blastocyst stage≥3, and at least one score of inner cell mass or trophectoderm is ≥B, according to Gardener scoring.

Embryos which have frozen or embryos transferred results in no pregnancy were excluded. PGT group were embryos which have CNV results by NGS. Medical records were those features in IVF treatments.

Live birth was defined as the birth of a live infant at ≥28 weeks of gestation. The live birth rate per embryo transfer was defined as the number of deliveries divided by the number of embryo transfers.

According to these criteria, totally 3469 static images and 154 time-lapse videos of embryos were collected from 543 patients and record features of these patients were analysis as well.

### Image Quality Control

During the image grading process, all images were first de-identified to remove any patient-related information. About 9% of the study participants were excluded due to poor photographic quality/unreadable images including: insufficient lighting such that the structures are clearly visible: sharp focus of the zona pellucida and trophectoderm; one embryo per micrograph with no visible instruments and little or no debris in the visual field: the entire embryo shown within the limits of the image (including the zona pellucida); and text or symbols in the images not hindering the visibility of the embryos.

Missing clinical diagnosis were also excluded. After establishing the consensus diagnoses, images were transferred to the AI team to develop a deep learning algorithm for image-based classification.

### Embryo Image Pre-Processing

The pre-processing of embryo image includes two steps, image segmentation and image enhancement.

Firstly, we cropped the embryo out of each image. We trained an embryo segmentation UNet³⁰ on embryo images to produce embryo segmentation masks, where the pixels on the embryo were assigned by positive labels (foreground) and the others were negative (background). These masks were used to locate the center of the embryo bounding box in each image. All embryo images were aligned by cropping along the calculated embryo bounding box. This alignment and cropping approach can help models to focus on the embryo in each image and reduce the bias introduced in the data collection stage.

To capture the non-specific features on embryo images and improve the performance of the AI models, two methods of image enhancement were utilized including Contrast Limited Adaptive Histogram Equalization (CLAHE)³¹ and color normalization³². Instead of globally performing histogram equalization, CLAHE enhancement was performed by dividing the image into local regions and applying histogram equalization over all neighborhood pixels. Compared with the original images, CLAHE enhanced the details of image. The image normalization method was performed as follows: x′=αx−αGauss(x,μ,Σ,s×s), where x is the input image, x′ is the normalized image, μ and β are parameters, and Gauss(x,μ,Σ,s×s) is a Gaussian filter with a Gaussian kernel (u,Σ) of size s×s. We used α=4 and β=128, Σ=I, and s=10, following the literature³². With image normalization, we could reduce the brightness bias among images taken under different acquisition conditions.

### Deep Learning and Transfer Learning Methods

Convolutional neural networks (CNNs) were used to analyze the embryo images in this study. The transfer learning technique was used, where the ResNet-50 model³³ pre-trained with the ImageNet dataset³⁴ was initialized as the backbone and fine-tuned for all deep learning models demonstrated. ResNet-50 is a five-stage network with residual designed blocks, which utilizes residual connections to overcome the degradation problem of deep learning models and enables very deep networks.

For the “regression” tasks, a fully connected layer with one scalar as output was used as the final layer in the ResNet-50 model. The final output was rounded to an integer for ordinal regression. For classification tasks, an additional softmax layer besides a fully connected layer was attached to the model.

The Mean-Square Error (MSE) loss was used as an objective function for “regression” tasks and the Cross Entropy loss was used for “classification” tasks. Embryo images were resized to 224×224. Training of models by back-propagation of errors was performed for 50 epochs with an Adam optimizer³⁵, learning rate of 10⁻³, weight decay of 10⁻⁶ and batch size of 32. Transformations of random horizontal flip, vertical flip, rotation and brightness were added to each batch during training as data augmentation in order to improve the generalization ability of the models. The models were implemented with PyTorch³⁶. We randomly divided the developmental dataset into a training set (⅞ of the development set) and a tuning set (⅛ of the development set) to develop our models. When training done, the models with the best validation loss were selected for evaluation on validation sets.

We applied model ensemble to improve the overall performance of the AI. For each task, we trained four model instances with different processed embryo images as input, where each input image was pre-processed into four images by applying CLAHE only, normalization only, both CLAHE and normalization, and identity transformation. Then, for each task, we trained four models with the same architecture trained in parallel on the same development set but with each using a differently pre-processed image. Given an input image, a prediction was obtained by averaging the outputs of the four models.

### Overview of the AI System

The disclosed AI system is a general embryo assessment platform covering the whole IVF/ICSI cycle, which include four main components: an embryo morphological grading module, a blastocyst formation assessment module, an aneuploid detection module, and a final live-birth occurrence prediction module.

AI models were first developed using multitask learning for embryo morphological assessment, including pronucleus type on day 1, and number of blastomeres, asymmetry and fragmentation of blastomeres on day 3.

### Embryo Morphological Grading and Multitask Learning

We built the embryo morphological grading module for embryo of day 1 and day 3, including evaluation of zona pellucida thickness variation, number of blastomeres, degree of cell symmetry and cytoplasmic fragmentation. We applied multitask learning for morphological grading of cleavage-stage embryos, since correlations between the morphology grades of cleavage-stage embryo are presented. For example, a cleavage-stage embryo shown with severe fragmentation is likely to consist of several asymmetrical blastomeres. Thus, we applied multitask learning for three tasks of morphology grading of cleavage-stage embryo to enhance the performance of the AI. The fragmentation rate and the number of cells were formulated to regression tasks and the identifying blastomere asymmetry was formulated to a binary classification task, whose loss functions were denoted as L_{f}, Lₙ, and Lₐ, respectively. A single model for these three different tasks was trained jointly through combining their loss functions, which not only could make use of the correlations but also performed regularization by sharing model parameters, resulting in more accurate and robust performance. We performed a homoscedastic uncertainty approach³⁷ to combining these three losses and minimized the joint loss. With the assumption of homoscedastic uncertainty, the loss of a task is weighted and factorized to

$$\frac{1}{{σ}^{2}} L + log σ$$

for a regression task or

$$\frac{1}{2 {σ}^{2}} L + log σ$$

for a classification task, where σ is a trainable parameter. Therefore, the combined loss function for the morphology grading multitask learning model can be formulated as

$$\frac{1}{2 {σ}_{f}^{2}} {L}_{f} + \frac{1}{2 {σ}_{n}^{2}} {L}_{n} + \frac{1}{2 {σ}_{a}^{2}} {L}_{a} + log {σ}_{f} + log {σ}_{n} + log {σ}_{a} .$$

### Blastocyst Formation Assessment and Noisy-or Inference

On the fifth day, the embryo forms a “blastocyst,” consisting of an outer layer of cells (the trophectoderm) enclosing a smaller mass (the inner-cell mass). In blastocyst formation assessment module, we used the embryo images from Day 1/Day 3 to predict the blastocyst formation. We trained two models for blastocyst formation assessment using embryos from Day 1 or Day 3, separately. We further combined the predicted results from these two models by a noisy-or inference, assuming that the development to blastocyst happening can be caused by two factors of embryo observed on Day 1 or Day 3, and the happening of any one of these two factors can lead to the happening of the blastocyst formation with independent probability. Thus, the probability of blastocyst formation is composited by p=1−Π_{i∈(1,3)}(1−pᵢ), where pᵢ is the predicted probability with the image on Day i.

We built an automatic evaluation system to detect embryo chromosomal ploidy and live birth outcome based on embryo still images and time-lapse videos. The embryo chromosomal ploidy (euploid vs. aneuploid) refers to the presence or absence of any wrong duplication or deletion of chromosomes, and the live-birth outcome refers to whether the embryo can be developed into a healthy fetus and delivered in a full term normally.

### Prediction of Chromosomal Ploidy Using Time-Lapse Image and Video

In the ploidy detection module, we adopted 3D neural networks to detect the embryo ploidy (euploid vs. aneuploid) based on the time-lapse video of the embryo development, which are images of embryos taken consecutively with the same time interval. Specifically, we uniformly sampled 128 frames per hour to capture the dynamic and static features of the embryos. And then we located the position of embryo using another neural network to align and size each and every embryo across all sampled time-lapse frames so each embryo image is uniform in size and pixels. We used a pretrained 3D ResNet to conduct the ploidy detection task based on the aligned embryo frames and gave the final prediction.

In an example, three-dimensional CNNs were adopted to predict the ploidy status (euploid vs aneuploid) of an embryo given an embryo time-lapse video, which presented both morphological and temporal information of the embryo³⁸. For each time-lapse video, firstly, we downsampled the frames of the video by uniformly sampling per hour with truncating or padding, resulting in a total of 128 frames, in order to capture morphological features and developmental kinetics of the embryo over the whole process of embryonic development. Then, the sampled images were cropped with the embryo segmentation model and resized to 128×128 for alignment. And then, the pre-processed images were stacked along temporal axis to generate a 128×128×128 3D tensor for downstream prediction tasks. We used a three-dimensional version of ResNet-18³⁹ model pre-trained with the Kinetics-400 dataset⁴⁰ to initialize the backbone and fine-tuned the classification head with embryo time-lapse videos for ploidy status prediction. The backbone consists of 3×3×3 and 3×7×7 convolutions, and the classification head consists of two fully connected layers. We used a five-fold cross-validation scheme for aneuploidy prediction.

### Live-Birth Prediction

In the live-birth prediction module, we used embryo images to predict the live-birth probability of a transfer with single or multiple embryos in a IVF transplantation. To improve the success rate of a single transfer therefore a high probability of a full term [pregnancy, multiple embryos are often transferred in a single transfer in practice. To address the variable length of the input data we built the neural network with CNN-RNN architecture. (CNN is the abbreviation of convolutional neural network, which is suitable for image feature extraction, and RNN is the abbreviation of recurrent neural network, which is designed for input data with a variable length). Image features of the embryos were extracted from each embryo in a single transfer by a shared CNN, and then further fused in the RNN to generate transfer-level feature, and finally aggregated to give an overall live-birth probability. Concretely, we used two views from day 1 and day 3 for each embryo. The input sequence was stacked embryo by embryo with ordered views along embryo developed time. We also integrate clinical metadata including maternal age, endometrial thickness, etc., to further improve prediction using methods such as logistic regression.

In an example, the live-birth occurrence prediction module mapped a transfer T with single or multiple embryos to a probability of live-birth occurrence, where T is a sequence of n×m images from n embryos with m viewed images. To address the input with variable numbers of embryo images in each transfer, we built the model M based on a feasible CNN-RNN architecture⁴¹, since CNNs were effective in extracting morphological features from embryo images, and recurrent neural networks (RNNs) were suitable for integrating information among embryo images. The model M consists of three parts: a CNN model Fᵥ, an RNN model Fₜ, and a two-layer perceptron classifier F_{c}. The CNN model Fᵥ extracts image-level feature eᵢ=Fᵥ(xᵢ) for each image xᵢ. We used the last flatten feature map produced by the backbone of Fᵥ as the input to the following RNN model. Then, the RNN model Fₜ with image features T=[x₁, x₂, . . . , xₙₘ] and an additional max-pooling layer over time axis will integrate the output of the RNN to a transfer-level feature f=Fₜ(T) with a fixed dimension for the following classification head. An additional max-pooling layer over time axis will integrate the output of the RNN to a transfer-level feature with a fixed dimension for the following classification head. The RNN model was implemented using a single layer bidirectional LSTM structure⁴². Finally, the two-layer perceptron classifier F_{c} map the transfer-level feature to the probability y=F_{c}(f). We used two views from day 1 and day 3 for each embryo. The input sequence was stacked embryo by embryo with ordered views along embryo developed time. We also combine/integrate clinical metadata to further improve prediction using methods such as logistic regression.

### Interpretation of AI Predictions

A SHAP method was used to display the impact of relevant risk factors on prediction for aneuploid detection and live-birth prediction. SHAP is a value explainable tool for tree-based models, which could efficiently and exactly compute local explanations and global explanations. The performance of a local explanation of SHAP for prediction with interpretability was also investigated.

In order to interpret the prediction that our models proposed, we used Integrated Gradient⁴³ (IG), a gradient-based method, to generate visual explanations that highlight areas contributing to the model's prediction. Given a trained model f, an input image x, and an output score y_{c}=f(x) for class c, the basic gradient-based visualization method⁴⁴ generates a saliency map where the importance weight for each pixel is derived by

$$\frac{\partial {y}_{c}}{\partial x} .$$

The IG method improves the basic method by path integrated gradients, which quantifies the importance of each pixel as follow

$$( x - {x}^{′} ) \times {\int }_{α = 0}^{1} \frac{\partial f ( {x}^{′} + α ( x - {x}^{′} ) )}{\partial x} d α ,$$

where x′ is a baseline image. This overcomes the disadvantage of the basic method that lacks sensitivity to important features when the model output to the correct class is saturated. In this study, the baseline image used a black image with the same size of input images. The generated heatmap was filtered by a Gaussian kernel with σ=8 for smooth.

### Performance Study of the AI System

To assess the ploidy predictions, AI system was compared against chance (randomly assigned ploidy predictions) and eight embryologists.

We conducted two experiments to study the AI system versus embryologist's performance in the ploidy evaluation. Given an embryo, we provided the images of Day 1, Day 3 and corresponding clinical metadata to the embryologists. The group of eight embryologists give a binary classification and a ranking evaluation of the data respectively.

In the binary classification evaluation experiment, the embryologists are asked to evaluate whether the embryo is euploid or not by looking at the picture and considering information provided for maternal information. For the AI's performance, we used the ROC evaluation and operating point-based binary classification, based on the generated probability.

For the ranking experiment, the embryologists assigned a score of 1 to 10, with the higher score indicating greater likelihood of euploidy. Each embryo was scored twice (two weeks after the initial reading) and the average was calculated as the final score. Further, we used the generated AI probabilities to calculate the ranking score for embryo evaluation and filtering for further PGT-A test. The euploidy rate of embryos is calculated at different filtering ratios.

### Statistical Analysis

To evaluate the performance of regression models for continuous values prediction in this study, we applied Mean Absolute Error (MAE), R-square (R2), and Pearson Correlation Coefficient (PCC). We applied the Bland-Altman plot +5 displaying the difference between the measured value and the predicted value of a sample against the average of the two. And we evaluated the agreement of the predicted value and actual value by 95% limits of agreement and Intraclass Correlation Coefficient (ICC). The models for binary classification were evaluated by Receiver Operating Characteristic (ROC) curves of sensitivity versus 1—specificity. The Area Under the Curve (AUC) of ROC curves were reported with 95% Confidence Intervals (CIs). The 95% CIs of AUC were estimated with the non-parametric bootstrap method (1,000 random resampling with replacement). The operating point of an Al system could be set differently to balance the true positive rate (TPR) and the false-positive rate (FPR). The embryo-level models were generated using the average outputs of predictions of image-level. The AUCs were calculated using the Python package of scikit-learn (version 0.22.1).

### Results

### Image Datasets and Patient Characteristics

After oocytes were retrieved, they were inseminated by conventional IVF according to sperm parameter. All the two-pronuclei embryos were cultured individually after fertilization check and were observed daily up to day-6. Each embryo had at least two photographs: one for fertilization check on Day-1 and one for Day-3 embryo morphological assessment. A total of 39,784 embryos from 7,167 patients were enrolled in the study which cultured from IVF/ICSI cycle between March 2010 and Dec. 31, 2018. The demographics and clinical information of the cohort participants are summarized in Table 1 and FIG. 8. Of those, 36,013 embryos from 6,453 patients were used as developmental dataset. All subjects from the developmental set were split randomly into mutually exclusive sets for training, tuning and “internal validation set” of the AI algorithm at a 70%:10%:20% ratio.

TABLE 1
Basic characteristics of patients in the developmental dataset and external
validation cohorts for diseases detection. The numbers of embryo images
used for identifying systemic conditions are shown in each cohort. AMH,
Anti-Müllerian hormone; FSH, Follicle-stimulating hormone.

| | Developmental Dataset | | | External | External |
|---|---|---|---|---|---|
| | Training | Tuning | Internal | validation | validation |
| Cohorts | set | set | validation | set 1 | set2 |
| Number of patients | 3,272 | 1,071 | 1,125 | 393 | 2,410 |
| Number of embryos | 9,141 | 3,130 | 3,331 | 407 | 3,192 |
| Number of images | 19,006 | 6,638 | 6,940 | 843 | 6,351 |
| Age (y), mean (SD) | 31.8 ± 4.4 | 31.9 ± 4.3 | 32.0 ± 4.6 | 31.5 ± 4.4 | 32.3 ± 4.8 |
| BMI (kg/m²), mean (SD) | 21.5 ± 2.9 | 21.4 ± 2.9 | 21.5 ± 2.9 | 21.4 ± 2.9 | 21.8 ± 3.1 |
| FSH (mIU/mL), mean (SD) | 6.0 ± 2.7 | 5.9 ± 2.1 | 5.9 ± 2.3 | 5.7 ± 1.9 | 6.8 ± 2.9 |
| AMH (ng/mL), mean (SD) | 5.1 ± 4.8 | 4.8 ± 3.9 | 4.8 ± 4.3 | 5.2 ± 4.2 | 3.9 ± 3.1 |
| Number of oocytes | 12.7 ± 7.1 | 12.8 ± 7.2 | 12.8 ± 6.9 | 13.1 ± 6.5 | 14.2 ± 8.7 |
| retrieved | | | | | |
| Endometrial thickness | 10.6 ± 2.8 | 10.4 ± 2.7 | 10.7 ± 2.6 | 10.7 ± 3.0 | 10.0 ± 1.8 |
| (mm), mean (SD) | | | | | |

In one embodiment of the present disclosure, the AI system provides a general embryo assessment platform covering the entire IVF/ICSI cycle, and include four modules: an embryo morphological grading module, a blastocyst formation assessment module, an aneuploid detection module, and a final live-birth occurrence prediction module.

AI models were first developed using multitask learning for embryo morphological assessment, including pronucleus type on day 1, and number of blastomeres, asymmetry and fragmentation rate of blastomeres on day 3. On the fifth day, the embryo forms a “blastocyst,” consisting of an outer layer of cells (the trophectoderm) enclosing a smaller mass (the inner-cell mass). We further used the embryo images from Day 1/Day 3 to predict the blastocyst formation with noisy-or inference (blastocyst formation assessment module).

The aneuploid detection module predicted the embryo ploidy (euploid vs. aneuploid) using embryo images and clinical metadata. We also constructed an 3D CNN model using time-lapse image videos and further tested on independent cohorts using videos from 400 patients to ensure the generalizability.

For the live-birth occurrence prediction module, embryo images and clinical metadata from 4,537 patients were used to train the AI model. To evaluate the AI model's performance, an independent prospective study was conducted. This prospective cohort consisted of 2,410 patients from Jiangmen hospital, Guangdong Province (Table 1, see more details in Methods).

### Explainable AI System for Embryo Morphological Assessment

In clinical practice, IVF embryos were selected for implantation according to a morphological score system at three stages, including pronuclei stage, cleavage stage, and blastocyst stage, according to the Istanbul consensus criteria.

Generally, the following parameters were used in the selection of the good quality embryos: pronuclear morphology, number of blastomeres at a particular day of culture: blastomere characteristics including size, symmetry and fragmentation.

At the pronuclei stage, the zygote (pronuclear) morphology has been related to the growth ability advancing to the blastocyst stage and to outcomes of implantation and pregnancy. The Z-score system was used to grade pronuclear of each embryo to Z1-Z4, in which nuclear size and alignment, nucleoli number and distribution are taken into account. The AI model was able to detect abnormal pronuclear morphology with an Area under the Curve (AUC) of 0.800 (95% CI: 0.783-0.814) (FIG. 2a).

At the cleavage stage, we evaluated the AI model ability to determine the asymmetry, fragmentation and number of blastomeres. Blastomere symmetry was defined as previously reported by Prados²⁰: embryos with blastomeres with a diameter difference of <25% were deemed symmetrical (−): embryos with ≥75% diameter differences were deemed severely asymmetrical (++), and a value between 25% and 75% was considered mildly symmetrical (+). This was calculated by dividing the diameter of the smallest blastomere with that of the largest blastomere (see more details in Methods). The AI system delivered an AUC of 0.817 (95% CI: 0.785-0.842) for the detection of the severe asymmetrical (++) from symmetrical blastomere, and an AUC of 0.870 (95% CI: 0.847-0.893) for the detection of asymmetrical (++ or +) from symmetrical blastomere (−) on test set (FIG. 2b).

We further compared between an AI predicted fragmentation score system and the actual fragmentation scoring system (FIG. 2c and FIG. 9a). The predicted and actual fragmentation of blastomeres had a strong linear relationship, with a Pearson correlation coefficient (PCC) of 0.86, coefficient of determination (R2) of 0.73, and a mean absolute error (MAE) of 3.335 percent (FIG. 2c). We then trained AI models to perform binary classification tasks (pattern of fragmentation versus normal). The AUC for detecting fragmentation was 0.971 (95% CI: 0.968-0.975) (FIG. 9a).

Lastly, we investigated the performance of the AI model to predict the cell numbers. FIG. 2d showed that the predicted cell numbers by Al algorithm achieved an excellent correlation with the actual number of blastomeres (PCC=0.863, R2=0.744, MAE=0.627).

### Prediction of Blastocyst Development Using Embryo Images

We next tested the ability of our AI models to predict the fate of cleavage-stage embryos. Accuracy of predicting stage of embryo development on D5 was established for D1 and D3 time points.

First, we investigated the performance by incorporating information from different time points including Day1/Day3 embryo images, using end-to-end deep learning methods (FIG. 3a). The AI model was able to predict whether or not an embryo could develop to the blastocyst stage with an AUC of 0.847 (95% CI: 0.838-0.856) using the Day 1 embryos alone. The AI model achieved an improved prediction accuracy with an AUC of 0.900 (95% CI:0.894-0.906) using the Day 3 embryos. When combined the Day 1 and Day 3 images, our model showed a better performance with an AUC of 0.913 (95% CI: 0.908-0.918).

We next assessed the ability for evaluation of embryo viability by using an embryo morphology scoring system from the previous study as input, which consisted of pronuclear morphology, asymmetry, fragmentation and number of blastomeres.

These studies demonstrated an improved predictive ability for evaluation of embryo viability when compared with embryologists' traditional morphokinetic grading methods (FIG. 10). Furthermore, fragmentation rate of embryos significantly increased with the failed blastocyst formation (FIG. 3b). Similarly, the asymmetry of embryos significantly increases with the failed blastocyst formation (FIG. 3c). FIG. 3d showed the examples that human blastocyst morphology including fragmentation and asymmetry of embryos, are correlated with the blastocyst development outcomes and were the main drivers of the overall AI assessment.

### Detection of Blastocyst Ploidy Using Embryo Image-Based AI System

Most of the embryos were selected to transfer according to morphological scores on day 3 or day 5, other embryos were transferred according to preimplantation genetic testing for aneuploidy (PGT-A) diagnosis reports. According to previous studies, embryo aneuploidies, which affect more than half of IVF embryos and increase with advancing maternal age, is the main reason for implantation failure²¹.

It is hypothesized that genome aneuploidy could affect cell morphology and migration patterns during embryonic development therefore amendable to detection by an AI algorithm. Three models were attempted for the aneuploidy detection: deep learning model using Day 1/Day 3 embryo images; baseline random forest model using clinical metadata: and a combined AI model using both input modalities. For all tasks, the combined model and the embryo image-only model performed better than the metadata-only model (FIG. 4a). The AUC for detecting embryo aneuploidies was 0.669 (95% CI: 0.641-0.702) for the metadata-only model, 0.719 (95% CI: 0.692-0.740) for the embryo image-only model and 0.785 (95% CI: 0.762-0.805) for the combined model (FIG. 4a).

Next, we first trained a 3D CNN model using time-lapse image video to predict the ploidy status (euploid vs aneuploid) of an embryo, which presented morphological and temporal information of the embryo development. The algorithm was further validated on a series of time-lapse video from 145 embryos. When tested on the external test set using still embryo images, the AUCs for predicting the presence of embryo aneuploidies were 0.648 (95% CI: 0.593-0.703) using a clinical metadata model, 0.740 (95% CI: 0.690-0.785) for an embryo image model, and 0.806 (95% CI: 0.760-0.837) for a combined model (FIG. 4b).

For interpreting the effects and relative contributions of the embryo features and clinical parameters on embryo aneuploidy prediction, we implemented an explainer SHAP (Shapley Additive explanation)²². The results showed that the embryo image features and clinical parameters including age, blastomere asymmetry, Day3 blastomere cell number are contributed to the prediction of aneuploid embryos (FIG. 4c).

We compared the performance between our AI system and eight embryologists from two different fertility clinics on aneuploidy prediction. In a euploidy screening setting, the embryologists ranked all the embryos for probability of being euploid. The top candidate embryos would be further selected to undergo a PGT-A testing. The testing dataset consisted of 560 images from 110 patients, from which 46.1% were euploid embryos. On this testing set, our AI system performance obtained an AUC of 0.724 which was superior overall to that of embryologists, including four junior embryologists and four senior embryologists (FIG. 4d).

Then we investigate whether our AI system could help embryologists to improve their performance for aneuploidy prediction. The embryologists were also asked to rank the embryos by looking at the pictures from embryos and considering information provided for maternal age and other clinical information (see more details in Methods).

We calculated the euploid rate with different selecting rate for further PGT-A testing and compare performance between our AI system and the embryologists (FIG. 4e). The baseline euploid rate of the population is 46.1%. By ordering the potential aneuploidy, the euploid rate by embryologist improved, and the AI-based performance was significantly improved compared to the embryologists. In addition, the euploid rate of embryos selected by our AI models would improve with the removal of the embryos increase.

### Predicting Live Birth Using Embryo Image and Clinical Metadata

To further extend the scope of our AI system in the prediction of live birth occurrence, we developed three models: baseline random forest models using clinical metadata: deep learning models using embryo images and a combined AI model using both input modalities. The developmental dataset was divided into training, tuning and internal validation sets (at a ratio of 7:1:2) to assess the models' performance (Data Table 1).

Here, the embryos were transferred on day 3 or day 5/6, and the number of embryos transferred to be limited to two or less embryos according to recent guidelines published in September 2004 by the American Society for Reproductive Medicine (ASRM)²³. Tested on the internal validation set, the clinical metadata alone gave an AUC of 0.722 (95% CI: 0.666-0.784), and the AI model trained using embryo images alone produced an AUC of 0.700 (95% CI: 0.636-0.751). When trained using combined clinical metadata and embryo images, the combined AI model achieved superior performance with an AUC of 0.803 (95% CI: 0.758-0.849) (FIG. 5a). We further validated these AI models using another independent external cohort (external validation set 1) to demonstrate their generalizability (FIG. 8). The AUC was 0.727 (95% CI: 0.657-0.798) for the clinical metadata-only model, 0.692 (95% CI: 0.604-0.759) for the embryo image model, and 0.762 (95% CI: 0.705-0.838) for the combined model (FIG. 5b).

Since the AI system measures many key embryo and key clinical features used in IVF, we further demonstrated it has the potential to reduce the time to grade embryos without sacrificing interpretability. Here, we used SHAP method to demonstrate the value of the explained predictions made by AI system and gain insight into factors that affect the live-birth occurrence. Our findings indicate that maternal age was identified as the most significant contributor in the clinical prognosis estimation. The maternal age, endometrial thickness, FSH, BMI, AMH are significantly associated with the live birth rate per transfer (FIG. 5c). Taken together, these findings demonstrated not only the validity the AI model, but also the potential real-life feasibility and utility of an AI-based platform.

### AI Assisted Live-Birth Prediction Performance Study

The embryos were selected for implantation according to morphological scores on day 3 or on day 5/6 based on a preimplantation genetic testing for aneuploidy (PGT-A) diagnosis report. To validate the AI system's clinical utility, we further studied the AI's performance on the external validation set 2 comprising 6,315 embryo images from 2,410 participants for scenario of single embryo transfer.

The performance of AI against embryologists in the live-birth rate on Day 3, or against live-birth results assisted by PGT-A on Day 5/6 have been summarized in FIG. 5d and FIG. 5e. For different clinical applications, the AI system's operating point can be set differently to compromise between the transfer rate and the live birth rate outcomes (FIG. 5d). Our baseline live-birth rate was 30.8% on Day 3 or 40.9% on Day 5, similar to the 29.3% or 45.0% reported in previous reference²⁴. When evaluated on the Day 3 transfer, our Al model achieved superior performance with live-birth of 46.0% compared to the baseline. Further, for the Day 5 transplant, the success rate of individual embryos by our AI model alone was 54.9% which was superior to that of PGT-A assisted performance (FIG. 5e). The results demonstrated that AI-assisted evaluation could help optimize embryo selection and maximizes likelihood of pregnancy with an accuracy comparable to that of a PGT-A test.

As the live-birth occurrence is correlated with age, we further analyzed our AI's performance in live-birth occurrence stratified by the median age (age-32). As shown in FIG. 11, the AI model had a significant 13.4% and 13.5% improvement compared to baseline on the older group (age>32), which is superior to that on the younger group (Age≤32).

### Visualization of Evidence for AI Prediction

Finally, to improve the interpretability of the AI model and shed light on its prediction mechanism, Integrated Gradients (IG) was used to generate saliency maps which help to highlight areas of the images that were important in determining the AI model's predictions. The saliency maps from the explanation techniques suggest that the model tends to focus on the pronuclear for evaluating the DI embryo morphology of pronuclear type (FIG. 6a).

As for the prediction of number of blastomeres and degree of cell symmetry, the model tends to focus on the spatial features around the center of D3 embryos (FIGS. 6b and 6d).

The knowledge derived from saliency maps appears to suggest that the AI model focuses on fragments around the cells of D3 embryos for cytoplasmic fragmentation and the fate of cleavage-stage embryos (failed one).

In FIG. 6e, the highlighted ‘points of interest’ map appears more scattered over the DI embryo that failed to develop to the cleavage-stage.

### Discussion

New progress in embryo selection aimed to maximize IVF success rates, reduce the time to conceive, while minimizing the risks of multiple pregnancies. Current morphological grading methods rely on descriptive parameters to rank cleavage-stage embryos for transfer. Though, previous studies have been studied for AI-assisted morphological grading²⁵ or identifying cleavage-stage embryos that will develop into blastocysts²⁶. This study has several differences to consider in comparison to previous studies.

In this study, we developed a general AI platform on the embryo evaluation and live-birth occurrence prediction for the entire IVF cycle, including an embryo morphological grading module, a blastocyst formation assessment module, an aneuploid detection module, and a final live-birth prediction module. The results raise the possibility of AI-based selection of embryos with manifestations beyond clinicians' observational power. These findings could potentially provide a non-invasive, high throughput and low-cost screening tool to greatly facilitate embryo selection and best outcome performance. It could also potentially assist in standardization of embryo selection methods across multiple clinical environments.

Oocyte²⁷ and embryo aneuploidies, affecting more than half of embryos produced and increasing with advancing maternal age, is the main reason for implantation failure and miscarriages in an IVF cycle, which was addressed by successful application of an IVF PGT-A test. However, this procedure is invasive and could cause embryo damages due to biopsy and vitrification: mis-diagnosis or mosaicism in PGT-A may result in embryo wastage: euploid assessment by NGS or SNP-array also means a higher cost in an IVF procedure.

Recently, the non-invasive strategy of time-lapse microscopy (TLM) was applied to human embryos and a lot of data analyzing the possible prognostic effect of morphokinetic were reported. Time-lapse microscopy evaluates the embryo quality by precise occurrence and duration of cell divisions (cytokinesis), duration of cell cycles (time interval between cleavages). Significant differences in morpho-kinetic pattern between euploid and aneuploid embryos may exist, but the clinical significance was absent to modest that are undetectable by human observers.

Here, our AI-based approach showed potential to extract morpho-kinetic parameters and be used as a surrogate for PGS to determine chromosomal status of the preimplantation embryos.

In addition, this study has assessed the role of automated AI algorithms in the live-birth rate using a D1/D3 embryo images and clinical metadata. And the selection accuracy was assessed for scenarios of single embryo transfers (SET) and double embryo transfers (DET). Our AI model showed much significant improvement compared to baseline live-birth rate. Though, the PGT-A achieved comparable performance with our AI-assisted approach, it has limitations that can only be used for blastocysts to transfer in Day 5. Further, our AI model can yield a continuous score that represents the quality of the embryo and that objective orders of transfer can be determined for a given set of embryos using such scores. For real-world clinical applications, the operating point of an AI system could be set differently to balance the transfer rate of blastocysts and the live-birth rate, which is more flexible compared to the PGT-A approach.

### REFERENCES

1. Baxter Bendus, A.E., Mayer, J.F., Shipley, S.K. & Catherino, W.H. Interobserver and intraobserver variation in day 3 embryo grading. Fertil Steril 86, 1608-1615 (2006).
2. Paternot, G., Devroe, J., Debrock, S., D'Hooghe, T.M. & Spiessens, C. Intra- and inter-observer analysis in the morphological assessment of early-stage embryos. Reprod Biol Endocrinol 7, 105 (2009).
3. Storr, A., Venetis, C.A., Cooke, S., Kilani, S. & Ledger, W. Inter-observer and intra-observer agreement between embryologists during selection of a single Day 5 embryo for transfer: a multicenter study. Hum Reprod 32, 307-314 (2017).
4. Rocha, J.C., et al. Automatized image processing of bovine blastocysts produced in vitro for quantitative variable determination. Sci Data 4, 170192 (2017).
5. Rocha, J.C., et al. A Method Based on Artificial Intelligence To Fully Automatize The Evaluation of Bovine Blastocyst Images. Sci Rep 7, 7659 (2017).
6. Topol, E.J. High-performance medicine: the convergence of human and artificial intelligence. Nat Med 25, 44-56 (2019).
7. Ravizza, S., et al. Predicting the early risk of chronic kidney disease in patients with diabetes using real-world data. Nat Med 25, 57-59 (2019).
8. Norgeot, B., Glicksberg, B.S. & Butte, A.J. A call for deep-learning healthcare. Nat Med 25, 14-15 (2019).
9. Esteva, A., et al. A guide to deep learning in healthcare. Nat Med 25, 24-29 (2019).
10. Kermany, D.S., et al. Identifying Medical Diagnoses and Treatable Diseases by Image-Based Deep Learning. Cell 172, 1122-1131 e1129 (2018).
11. Liang, H., et al. Evaluation and accurate diagnoses of pediatric diseases using artificial intelligence. Nat Med 25, 433-438 (2019).
12. Wang, C., Elazab, A., Wu, J. & Hu, Q. Lung nodule classification using deep feature fusion in chest radiography. Comput Med Imaging Graph 57, 10-18 (2017).
13. Khosravi, P., et al. Deep learning enables robust assessment and selection of human blastocysts after in vitro fertilization. NPJ Digit Med 2, 21 (2019).
14. Kanakasabapathy, M.K., et al. Development and evaluation of inexpensive automated deep learning-based imaging systems for embryology. Lab Chip 19, 4139-4145 (2019).
15. Dimitriadis, I., et al. Automated smartphone-based system for measuring sperm viability, DNA fragmentation, and hyaluronic binding assay score. PLOS One 14, e0212562 (2019).
16. Bormann, C.L., et al. Performance of a deep learning based neural network in the selection of human blastocysts for implantation. Elife 9(2020).
17. Wahl, B., Cossy-Gantner, A., Germann, S. & Schwalbe, N.R. Artificial intelligence (AI) and global health: how can AI contribute to health in resource-poor settings? BMJ Glob Health 3, e000798 (2018).
18. Hosny, A. & Aerts, H. Artificial intelligence for global health. Science 366, 955-956 (2019).
19. Goyal, A., Kuchana, M. & Ayyagari, K.P.R. Machine learning predicts live-birth occurrence before in-vitro fertilization treatment. Scientific reports 10, 1-12 (2020).
20. Prados, F.J., Debrock, S., Lemmen, J.G. & Agerholm, I. The cleavage stage embryo. Human Reproduction 27, 150-171 (2012).
21. Fragouli, E., et al. The origin and impact of embryonic aneuploidy. Human genetics 132, 1001-1013 (2013).
22. Lundberg, S.M., Erion, G.G. & Lee, S.-I. Consistent individualized feature attribution for tree ensembles. arXiv preprint arXiv: 1802.03888 (2018).
23. Technology, P.C.o.t.S.f.A.R. Guidelines on the number of embryos transferred. Fertility and sterility 82, 1-2 (2004).
24. Kamath, M.S., Mascarenhas, M., Kirubakaran, R. & Bhattacharya, S. Number of embryos for transfer following in vitro fertilisation or intra-cytoplasmic sperm injection. Cochrane Database of Systematic Reviews (2020).
25. Leahy, B.D., et al. Automated Measurements of Key Morphological Features of Human Embryos for IVF. in International Conference on Medical Image Computing and Computer-Assisted Intervention 25-35 (Springer, 2020).
26. Thirumalaraju, P., et al. Deep learning-enabled blastocyst prediction system for cleavage stage embryo selection. Fertility and sterility 111, e29 (2019).
27. Minasi, M.G., et al. Correlation between aneuploidy, standard morphology evaluation and morphokinetic development in 1730 biopsied blastocysts: a consecutive case series study. Human Reproduction 31, 2245-2254 (2016).
28. Scott, L., Alvero, R., Leondires, M. & Miller, B. The morphology of human pronuclear embryos is positively related to blastocyst development and implantation. Human reproduction 15, 2394-2403 (2000).
29. The Istanbul consensus workshop on embryo assessment: proceedings of an expert meeting. Human reproduction 26, 1270-1283 (2011).
30. Ronneberger, O., Fischer, P. & Brox, T. U-net: Convolutional networks for biomedical image segmentation. in International Conference on Medical image computing and computer-assisted intervention 234-241 (Springer, 2015).
31. Pisano, E.D., et al. Contrast limited adaptive histogram equalization image processing to improve the detection of simulated spiculations in dense mammograms. Journal of Digital imaging 11, 193 (1998).
32. Graham, B. Kaggle diabetic retinopathy detection competition report. University of Warwick (2015).
33. He, K., Zhang, X., Ren, S. & Sun, J. Deep residual learning for image recognition. in Proceedings of the IEEE conference on computer vision and pattern recognition 770-778 (2016).
34. Deng, J., et al. Imagenet: A large-scale hierarchical image database. in 2009 IEEE conference on computer vision and pattern recognition 248-255 (Ieee, 2009).
35. Kingma, D.P. & Ba, J. Adam: A method for stochastic optimization. arXiv preprint arXiv: 1412.6980 (2014).
36. Paszke, A., et al. Pytorch: An imperative style, high-performance deep learning library. arXiv preprint arXiv: 1912.01703 (2019).
37. Kendall, A., Gal, Y. & Cipolla, R. Multi-task learning using uncertainty to weigh losses for scene geometry and semantics. in Proceedings of the IEEE conference on computer vision and pattern recognition 7482-7491 (2018).
38. Gardner, D.K., Meseguer, M., Rubio, C. & Treff, N.R. Diagnosis of human preimplantation embryo viability. Human reproduction update 21, 727-747 (2015).
39. Tran, D., et al. A closer look at spatiotemporal convolutions for action recognition. in Proceedings of the IEEE conference on Computer Vision and Pattern Recognition 6450-6459 (2018).
40. Kay, W., et al. The kinetics human action video dataset. arXiv preprint arXiv: 1705.06950 (2017).
41. Yue-Hei Ng, J., et al. Beyond short snippets: Deep networks for video classification. in Proceedings of the IEEE conference on computer vision and pattern recognition 4694-4702 (2015).
42. Hochreiter, S. & Schmidhuber, J. Long short-term memory. Neural computation 9, 1735-1780 (1997).
43. Sundararajan, M., Taly, A. & Yan, Q. Axiomatic attribution for deep networks. in International Conference on Machine Learning 3319-3328 (PMLR, 2017).
44. Simonyan, K., Vedaldi, A. & Zisserman, A. Deep inside convolutional networks: Visualising image classification models and saliency maps. arXiv preprint arXiv: 1312.6034 (2013).
45. Giavarina, D. Understanding bland altman analysis. Biochemia medica 25, 141-151 (2015).

All publications, patents, and patent applications mentioned in this specification are herein incorporated by reference to the same extent as if each individual publication, patent, or patent application was specifically and individually indicated to be incorporated by reference.

1. A computer-implemented method comprising the steps of:
receiving image data of one or more human embryos, the image data including a plurality of images of the one or more human embryo at different time points within the first 6 days after the formation of the one or more embryos;
determining a viability indicator for the one or more human embryos, wherein the viability indicator represents a likelihood that selection for implantation of the one or more embryos will result in a viable embryo, based on one or more the following:
determining embryo morphological grading of the one or more embryos using a first neural network based on the image data;
determining aneuploidy of the one or more embryos using a second deep learning model at least partly based on the image data;
predicting live-birth occurrence of a transfer of the one or more embryos for implantation using a third deep learning model at least partly based on the image data; and
outputting the viability indicator.

2. The method of claim 1, wherein determining the embryo morphological grading comprises using a multitask machine learning model based on the following three tasks: (1) a regression task for the cytoplasmic fragmentation rate of the embryo, (2) a binary classification task for the number of cells of the embryo, and (3) a binary classification task for the blastomere asymmetry of the embryo determined, based on the image data.

3. The method of claim 2, wherein the multitask machine learning model was trained jointly through combining the loss functions of the three tasks by using a homoscedastic uncertainty approach in minimizing the joint loss.

4. The method of claim 1, wherein output parameters for the embryo morphological grading comprise pronucleus type on Day 1, the number of blastomeres, asymmetry, and fragmentation of blastomeres on Day 3.

5. The method of claim 1, wherein determining a viability indicator for the human embryo further comprises using clinical metadata of the donor of the egg from the embryo is developed, the metadata includes at least one of maternal age, menstrual status, uterine status, and cervical status, previous pregnancy, and fertility history.

6. The method of claim 1, wherein the second deep learning model in the aneuploidy determination is a 3D CNN model trained by time-lapse image videos and PGT-A based ploidy outcomes assessed by biopsy.

7. The method of claim 1, further comprising:
determining blastocyst formation based on the embryo image data based on Day 1 and Day 3.

8. The method of claim 1, wherein the third deep learning model comprises a CNN model.

9. The method of claim 1, further including: determining a ranking of a plurality of human embryos based on their viability indicators.

10. The method of claim 9, further including: selecting, based on the ranking, one of the plurality of human embryos for a single embryo transfer or the order in which multiple embryos should be transferred.

11. The method of claim 1, further comprising: selecting the embryo for transfer and implantation based on the determined viability indicator.

12. The method of claim 11, wherein the selection for transfer and implantation is on Day 3, Day 5 or Day 6.

13. The method of claim 1, wherein determining the viability indicator comprises determining aneuploidy of the one or more embryos using the second deep learning model at least partly based on the image data.

14. The method of claim 13, wherein determining aneuploidy of the one or more embryos comprises using 3D neural networks.

15. The method of claim 13, wherein determining aneuploidy of the one or more embryos comprises using time-lapse video of embryo development and normalizing all images in the time-lapse video with the same size and number of pixels.

16. The method of claim 1, wherein determining the viability indicator comprises predicting live-birth occurrence of a transfer of the one or more embryos for implantation using the third deep learning model at least partly based on the image data.

17. The method of claim 16, wherein predicting live-birth occurrence of a transfer of the one or more embryos for implantation comprises utilizing a CNN architecture to produce an overall live-birth probability.

18. A method of selecting a human embryo in an IVF/ICSI cycle, comprising:
determining a viability indicator using a computer-implemented prediction method of any of claim 1;
based on the predicted viability indicator, selecting the human embryo for transfer and implantation.

19. A system, including at least one processor configured to:
receive image data of one or more human embryos, the image data including a plurality of images of the one or more human embryos with at different time points within the first 6 days after the formation of the one or more embryos;
apply at least one three-dimensional (3D) artificial neural network to the image data to determine a viability indicator for the one or more human embryos; and
output the viability score;
wherein the viability indicator represents a likelihood that the one or more embryos will result in at least one viable embryo;
wherein determining the viability indicator for the one or more human embryos comprises at least one of:
determining embryo morphological grading of the one or more embryos using a first neural network based on the image data;
determining aneuploidy of the one or more embryos using a second deep learning model at least partly based on the image data; and
predicting live-birth occurrence of a transfer of the one or more embryos for implantation using a third deep learning model at least partly based on the image data.

20. The system of claim 19, wherein determining the embryo morphological grading comprises using a multitask machine learning model based on the following three tasks: (1) a regression task for the cytoplasmic fragmentation rate of the embryo, (2) a binary classification task for the number of cells of the embryo, and (3) a binary classification task for the blastomere asymmetry of the embryo determined, based on the image data.

21.-24. (canceled)
